(19) United States
(12) Patent Application Publication
(10) Pub. No.: US 2021/0068635 A1
(43) Pub. Date: Mar. 11, 2021

(54) ENDOSCOPE APPARATUS

(71) Applicant: OLYMPUS CORPORATION, Tokyo (JP)

(72) Inventors: Momoko Yamanashi, Tokyo (JP); Kazuhiko Hino, Tokyo (JP)

(73) Assignee: OLYMPUS CORPORATION, Tokyo (JP)

(21) Appl. No.: 17/016,853

(22) Filed: Sep. 10, 2020

Related U.S. Application Data
(63) Continuation of application No. PCT/JP2019/002996, filed on Jan. 29, 2019.

(30) Foreign Application Priority Data
Mar. 14, 2018 (JP) 2018-047034

(51) Int. Cl. A61B 1/015; A61B 1/00

(52) U.S. Cl. CPC A61B 1/015; A61B 1/00006; A61B 1/00068

(57) ABSTRACT

An endoscope apparatus includes an insertion portion, a liquid feeding apparatus, an air feeding apparatus, a third flow path, a joining portion, a processor, a distal end portion including an opening from which liquid and air are ejected, and a valve configured to perform a switching operation of flowing and cutoff of the air in the second flow path under operation control of the processor such that the liquid and the air alternately flow in the third flow path.

### CROSS REFERENCE TO RELATED APPLICATION

This application is a continuation application of PCT/JP2019/002996 filed on Jan. 29, 2019 and claims benefit of Japanese Application No. 2018-047034 filed in Japan on Mar. 14, 2018, the entire contents of which are incorporated herein by this reference.

### BACKGROUND OF THE INVENTION

### 1. Field of the Invention

The present invention relates to an endoscope apparatus that ejects liquid and air from an opening of a distal end portion of an insertion portion into a subject or object in a longitudinal direction.

### 2. Description of the Related Art

In recent years, endoscopes have been widely used in medical and industrial fields. The endoscope can observe the inside of the subject or object by an insertion of an elongated insertion portion into the subject or object.

In order to secure a good observation field of view in the subject or object, a configuration is well known in which a fluid can be supplied, in a state where the insertion portion is inserted into the subject or object, from an opening of a fluid supply conduit formed at a distal end portion located on a distal end side (hereinafter, simply referred to as a distal end side) in a longitudinal axis direction of the insertion portion toward a point in the longitudinal axis direction, that is, toward a front in the longitudinal axis direction from the opening.

Specifically, a configuration of an endoscope apparatus is well known in which a fluid flows from a fluid supply apparatus to the fluid supply conduit provided in an endoscope and the fluid is supplied to contaminations adhering to a wall surface of a subject or object from an opening of the fluid supply conduit, so that the contaminations can be removed from the wall surface. Examples of the fluid include air, liquid, and a mixture of air and liquid.

Further, a configuration of an endoscope apparatus is also well known in which a suction conduit connected with a suction apparatus and provided in an endoscope is used to suck contaminations removed from a wall surface by supplying a fluid through an opening of the suction conduit formed at a distal end portion.

Here, for example, when an insertion portion is inserted into a large intestine using, for example, a medical endoscope to observe a subject, a large amount of residue may remain in the large intestine because the subject may forget to take a laxative or may not comply with dietary restrictions, or cases of emergency disease may occur.

In this case, using the endoscope apparatus described above, first, contaminations on a mucous membrane in the large intestine are removed by repetition of fluid supply and residue suction, and then inspection or treatment is performed.

Therefore, a configuration of an endoscope apparatus is also well known in which liquid and air are simultaneously ejected to residues from separate conduits to supply a mist-like fluid, so that a supply pressure of the fluid is increased compared with a case where only air or liquid is supplied and thus the residue can be removed in a short time.

Further, Japanese Patent Application Laid-Open Publication No. H6-14870 discloses an endoscope apparatus in which an air-water mixed fluid formed by a mixture of air and liquid is supplied to residues and a collision property of the liquid with respect to the residue is enhanced by the air, so that the residues are further removed compared with the case where the mist-like fluid is supplied.

### SUMMARY OF THE INVENTION

An endoscope apparatus according to an aspect of the present invention includes: an insertion portion configured to be inserted into a subject from a distal end side in a longitudinal direction; a liquid feeding apparatus configured to cause a liquid to flow in a first flow path; an air feeding apparatus configured to cause air to flow in a second flow path; a third flow path provided at least partially in the insertion portion and configured to cause the first flow path and the second flow path to communicate with each other; a joining portion in which the air flowing in the second flow path intermittently flows to the liquid flowing in the first flow path with a timing in which the air is not mixed with the liquid, the joining portion of the first flow path and the second flow path being formed in the third flow path; a processor configured to control an operation of the liquid feeding apparatus and the air feeding apparatus such that the liquid and the air flow alternately in the third flow path through the joining portion; a distal end portion provided at a distal end of the insertion portion in the longitudinal direction and including an opening from which the liquid and the air flowing alternately in the third flow path are ejected into the subject in the longitudinal direction; and a valve configured to perform a switching operation of flowing and cutoff of the air in the second flow path under operation control of the processor such that the liquid and the air alternately flow in the third flow path.

### BRIEF DESCRIPTION OF THE DRAWINGS

FIG. 1 is a view schematically showing an endoscope apparatus of a first embodiment;

FIG. 2 is a view schematically showing a valve provided in a second flow path in the endoscope apparatus of FIG. 1 and the second flow path;

FIG. 3 is a view schematically showing a state where air flowing in the second flow path alternately flows in a liquid flowing in a first flow path in a third flow path in the endoscope apparatus of FIG. 1;

FIG. 4 is a view schematically showing a state where the liquid ejected from an opening of the third flow path of FIG. 1 collides with residues due to a supply pressure being increased by the air;

FIG. 5 is a view schematically showing a modification in which a switch is electrically connected to a processor of FIG. 1 to instruct an opening/closing timing of the valve;

FIG. 6 is a view schematically showing a modification in which the valve of FIG. 1 is provided at a joining portion;

FIG. 7 is a view schematically showing a modification in which the joining portion of FIG. 1 is provided in an operation portion;

FIG. 8 is a view schematically showing a modification in which the joining portion of FIG. 1 is provided outside an endoscope;

FIG. 9 is a view schematically showing a modification in which the valve of FIG. 1 is formed by two disks, and the second flow path;

FIG. 10 is an enlarged plan view of a fixed disk of FIG. 9;

FIG. 11 is an enlarged plan view of a rotating disk of FIG. 9;

FIG. 12 is a view schematically showing a modification in which the valve of FIG. 1 is formed only by the rotating disk of FIG. 11, and the second flow path;

FIG. 13 is a plan view showing a modification of the rotating disk of FIG. 11;

FIG. 14 is a view schematically showing a modification in which the valve of FIG. 1 is configured to open and close by moving up and down with respect to the second flow path 12, and the second flow path;

FIG. 15 is a view schematically showing a modification in which the valve of FIG. 1 is provided with a pressurizing unit, and the second flow path and the processor;

FIG. 16 is a view schematically showing a configuration of an endoscope apparatus of a second embodiment;

FIG. 17 is a chart showing liquid feeding and water feeding timing by a flow timing adjusting unit of FIG. 16;

FIG. 18 is a chart showing a modification of liquid feeding and water feeding timing by the flow timing adjusting unit in FIG. 17; and

FIG. 19 is a view schematically showing a modification of the endoscope apparatus in which a pressurizing unit is provided in an external apparatus of FIG. 16.

### DETAILED DESCRIPTION OF THE PREFERRED EMBODIMENTS

Embodiments of the present invention will be described below with reference to the drawings. Note that the drawings are schematic, the relation between a thickness and a width of each member and the ratio of the thickness of each member are different from actual relation and ratio, and components having different dimensional relations and ratios are naturally included.

In embodiments to be described below, an endoscope apparatus will be described by taking a medical endoscope apparatus as an example.

### First Embodiment

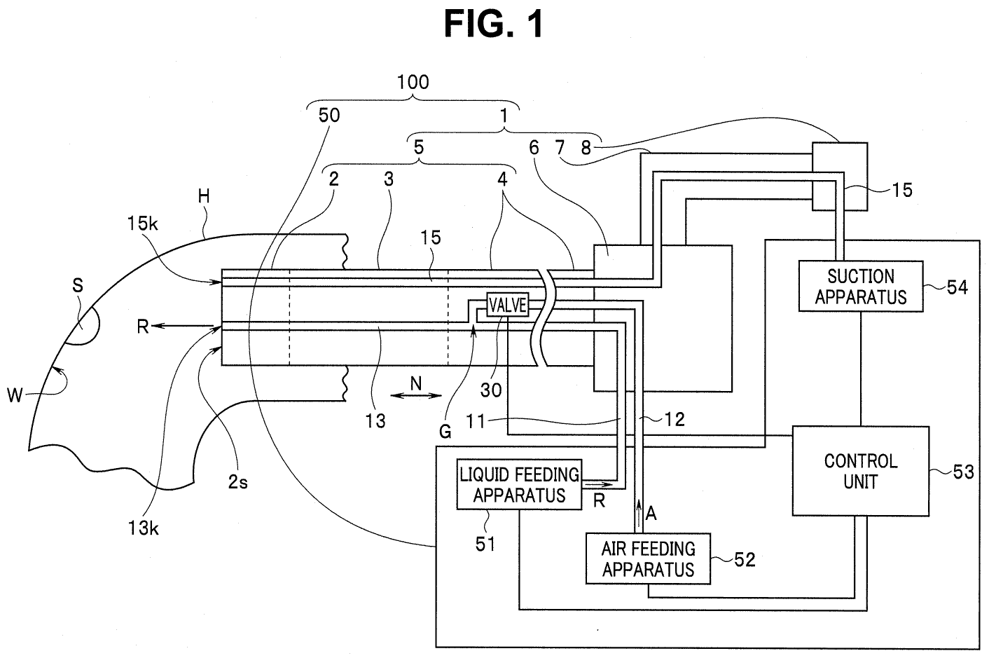

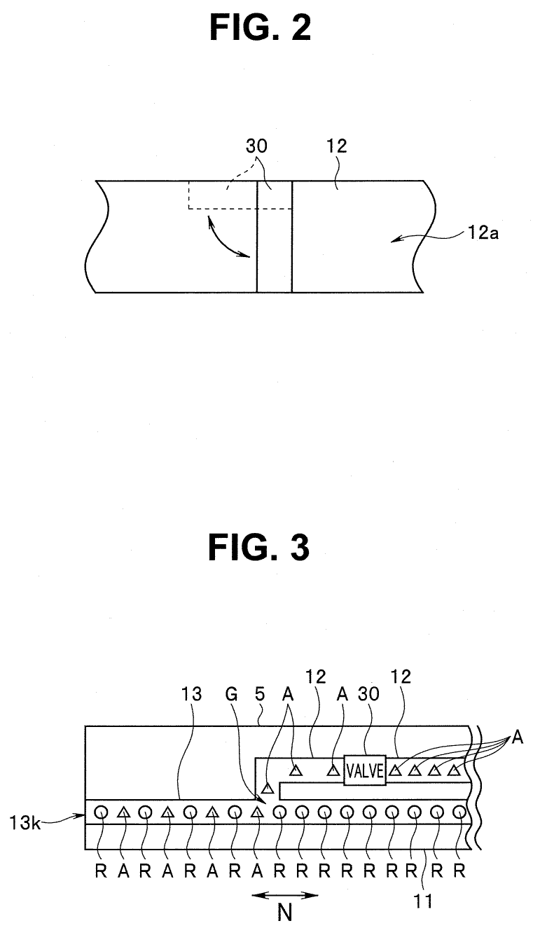

FIG. 1 is a schematic view of an endoscope apparatus according to the present embodiment, and FIG. 2 is a view showing schematically a second flow path and a valve provided in the second flow path in the endoscope apparatus shown in FIG. 1.

Further, FIG. 3 is a view schematically showing a state where air flowing in the second flow path alternately flows in a liquid flowing in a first flow path in a third flow path in the endoscope apparatus of FIG. 1

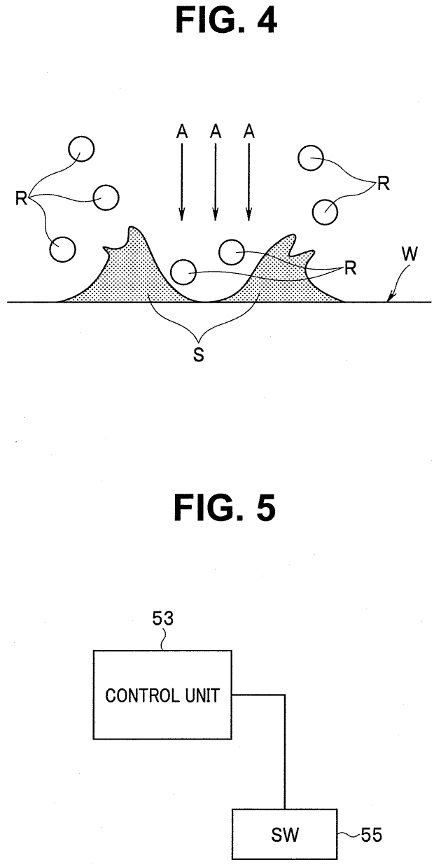

Further, FIG. 4 is a view schematically showing a state where the liquid ejected from an opening of the third flow path of FIG. 1 collides with residues due to a supply pressure being increased by the air, and FIG. 5 is a view schematically showing a modification in which a switch is electrically connected to a processor of FIG. 1 to instruct an opening/closing timing of the valve.

As shown in FIG. 1, an endoscope apparatus 100 includes an endoscope 1 and a peripheral apparatus 50.

The endoscope 1 includes an insertion portion 5 inserted into, for example, a large intestine H, which is a subject, from a distal end side, and an operation portion 6 connected to a proximal end of the insertion portion 5 in a longitudinal direction N.

In addition, the endoscope 1 includes a universal cord 7 extending from the operation portion 6 and a connector 8 provided at an extension end of the universal cord 7.

The insertion portion 5 includes a distal end portion 2 provided on a distal end side, a bending portion 3 provided on a proximal end side of the distal end portion 2 in the longitudinal direction N, and a flexible tube portion 4 provided on a proximal end side of the bending portion 3 in the longitudinal direction N, and is formed in an elongated shape.

The peripheral apparatus 50 includes a liquid feeding apparatus 51, an air feeding apparatus 52, a processor 53, and a suction apparatus 54.

The liquid feeding apparatus 51 is configured to operate under a drive control of the processor 53. In the endoscope 1 of the present embodiment, the liquid feeding apparatus 51 causes a liquid R to flow through a first flow path 11 provided in the operation portion 6 and the insertion portion 5 in part, specifically, in the flexible tube portion 4, as shown in FIG. 3.

The air feeding apparatus 52 is configured to operate under the drive control of the processor 53. In the present embodiment, the air feeding apparatus 52 causes air A to flow through a second flow path 12 provided in the operation portion 6 and the insertion portion 5 in part, specifically, in the flexible tube portion 4, as shown in FIG. 3.

In the present embodiment, a third flow path 13, in which the first flow path 11 and the second flow path 12 are joined at a joining portion G, is provided in the insertion portion 5.

The joining portion G is located near the bending portion 3 inside the flexible tube portion 4. Further, a distal end of the third flow path 13 in the longitudinal direction N is opened as an opening 13k in a distal end surface 2s of the distal end portion 2.

The processor 53 controls the operation of the liquid feeding apparatus 51 and the air feeding apparatus 52 such that the liquid R and the air A flow alternately in layers in the third flow path 13 through the joining portion G.

In the joining portion G, as shown in FIG. 3, the air A flowing in the second flow path 12 intermittently flows to the liquid R flowing in the first flow path 11, with a timing in which the air A is not mixed with the liquid R, under opening/closing control of a valve 30 by the processor 53.

The valve 30 is provided at a halfway position of the second flow path 12 to perform a switching operation of flowing and cutoff of the air A in the second flow path 12 by the operation control of the processor 53.

Thus, the valve 30 switches a time during which the liquid R flows from the first flow path 11 to the third flow path 13 through the joining portion G and a time during which the air A flows from the second flow path 12 to the third flow path 13 through the joining portion G at a predetermined timing, thereby causing the liquid R and the air A to flow alternately in layers in the third flow path 13.

Further, the valve 30 has an opening/closing valve structure as shown in FIG. 2, for example.

The valve 30 may be opened and closed intermittently under electrical control of the processor 53. In addition, the valve 30 may be opened when a pressure of the air A flowing from the air feeding apparatus 52 to the second flow path 12 in a space 12a (see FIG. 2) formed on an upstream side of the valve 30 is equal to or higher than a threshold value.

Further, the valve 30 may be opened and closed intermittently for a constant period of time under the electric control of the processor 53.

In addition, as shown in FIG. 5, the valve 30 may be opened and closed intermittently for a constant period of time by an operation of a switch (SW) 55, which is electrically connected to the processor 53, from an operator.

An example of the switch 55 may include a foot switch, a dial switch, or a push switch.

An opening/closing interval of the valve 30 is set to a value desired by the operator by an input from the switch 55 or a change of the threshold value of the pressure of the air A in the space 12a described above.

In other words, as the opening/closing interval of the valve 30 becomes shorter, a ratio of the air A and the liquid R flowing alternately in layers in the third flow path 13 for a constant period of time becomes larger. As the opening/closing interval of the valve 30 becomes longer, a ratio of the air A and the liquid R flowing alternately in layers in the third flow path 13 for a constant period of time becomes smaller.

From the opening 13k, as shown in FIGS. 1 and 3, the liquid R and the air A, which alternately flow in layers in the third flow path 13, are ejected into the large intestine H in the longitudinal direction N.

Specifically, from the opening 13k, the liquid R and the air A flowing alternately in layers in the third flow path 13 are ejected toward contaminations adhering to an intestinal wall W of the large intestine H, for example, a residue S, that is, forward in the longitudinal direction N as shown in FIGS. 1 and 4.

The reason to alternately flow the liquid R and the air A in layers in the third flow path 13 and to eject the liquid R and the air A from the opening 13k is as follows.

When the air A is sandwiched between the liquids R in the longitudinal direction N in the third flow path 13, the liquid R is compressed by the air A to form a mass of the high-pressure liquid R, the mass is supplied toward the residue S from the opening 13k as shown in FIG. 4 with a high supply pressure, and thus a collision force of the liquid R is increased with respect to the residue S with a small amount of liquid to be fed and a cleaning power is increased.

This point is different from a conventionally used configuration in which a mist-like liquid is supplied to the residue S as described above or a configuration in which a fluid formed by mixing of air with a liquid is simply supplied. In other words, the collision force of the liquid R with respect to the residue S is increased compared with the conventional configuration.

Although not shown, an aperture may be detachably attached to the opening 13k to increase an ejecting force of the mass of the liquid R with respect to the residue S or to expand an ejecting range.

As described above, the reason why the joining portion G is provided near the bending portion 3 in the flexible tube portion 4 is that when the air A is mixed with the liquid R at a position as close to the opening 13k as possible, the mass of the liquid R compressed by the air A is ejected from the opening 13k in a state where the supply pressure is high, that is, the collision force against the residue S is increased.

Therefore, the joining portion G is preferably located as close to the opening 13k as possible to increase the collision force of the mass of the liquid R.

However, when the joining portion G is provided in the distal end portion 2, two flow paths, for example, the first flow path 11 and the second flow path 12 are provided in the distal end portion 2 and the bending portion 3, and thus the distal end portion 2 and the bending portion 3 are increased in diameter.

Further, even when the joining portion G is provided in the bending portion 3, since the two flow paths 11 and 12 are provided in the bending portion 3, the bending portion 3 is increased in diameter.

Further, when the two flow paths 11 and 12 and the joining portion G are provided in the bending portion 3, the rate of members to be filled in the bending portion 3 increases compared with a case where one flow path is provided or a case where the joining portion G is not provided. For this reason, the resistance of the bending portion 3 is lowered, and the amount of force for bending the bending portion 3 is increased.

Accordingly, in the present embodiment, one flow path configured to supply the liquid R is provided in the distal end portion 2 and the bending portion 3. Further, the joining portion G is provided near the bending portion 3 in the flexible tube portion 4 to increase the collision force of the mass of the liquid R as much as possible without hindering bending performance of the bending portion 3 in the state where the diameters of the distal end portion 2 and the bending portion 3 are maintained.

Further, a suction conduit 15 including an opening 15k in the distal end surface 2s is provided in the insertion portion 5, the operation portion 6, the universal cord 7, and the connector 8.

The suction apparatus 54 operates under the drive control of the processor 53, and is connected to the suction conduit 15 to suck the residue S, which is removed from the intestinal wall W of the large intestine H by the supply of the mass of the liquid R, through the suction conduit 15.

Other components of the endoscope apparatus 100 are equal to the components of the convention endoscope apparatus, and thus will not be described.

As described above, an operation control of the processor 53 when the mass of the liquid R is supplied to the residue S using the endoscope apparatus 100 configured as described above will be briefly described below.

First, the liquid R is caused to flow in the first flow path 11 from the liquid feeding apparatus 51, and then the air A is caused to flow in the second flow path 12 from the air feeding apparatus 52.

Then, the operation of the liquid feeding apparatus 51 and the air feeding apparatus 52 is controlled such that the liquid R and the air A flow alternately in the third flow path 13, that is, in the present embodiment, controls the opening/closing of the valve 30 such that the liquid R and the air A flow alternately in the third flow path 13.

Thereby, in the joining portion G, the air A flowing in the second flow path 12 intermittently flows to the liquid R flowing in the first flow path 11 with a timing in which the air A is not mixed with the liquid R.

As a result, the liquid R and the air A alternately flowing in the third flow path 13 are ejected from the opening 13k toward the residue S.

As described above, the case is described in the present embodiment in which the valve 30 causes the liquid R and the air A to alternately flow in layers into the third flow path 13 from the first flow path 11 and the second flow path 12 through the joining portion G, and the mass of the high-pressure liquid R compressed by the air A is ejected from the opening 13k.

According to the present embodiment, since the mass of the liquid R can be supplied to the residue S with a high collision force, the residue S sticking to the intestinal wall W of the large intestine H can be reliably removed in a short time even with a small amount of liquid compared with the conventional mist-like liquid or the fluid in which the air is simply mixed with the liquid.

In addition, since the cleaning power is improved, the amount of the liquid R supplied into the large intestine H may be small, so that suction work of the liquid R and the residue S through the suction conduit 15 after the liquid R is supplied can also be performed in a short time.

Further, since the joining portion G is provided in the flexible tube portion 4, it is not necessary to provide two flow paths through which the liquid R for removing the residue S in the distal end portion 2 and the bending portion 3. In other words, one flow path may be provided.

As described above, the diameters of the distal end portion 2 and the bending portion 3 can be maintained as in the conventional endoscope apparatus, and the joining portion G is located near the bending portion 3 in the flexible tube portion 4, so that the mass of the liquid R can be supplied to the residue S with a higher supply pressure.

In order to increase the supply pressure of the fluid supplied to the residue S, it is not necessary that an air supply conduit and a liquid supply conduit are increased in diameter, or a thick conduit having pressure resistance is used for these conduits, or the sizes of the air feeding apparatus and the liquid feeding apparatus are increased to increase a supply capacity. Thus, it is possible to prevent the insertion portion 5 from being larger in diameter and the endoscope apparatus 100 from being larger in size.

As described above, it is possible to provide the endoscope apparatus 100 having a configuration capable of removing the contaminations with a small amount of fluid to be supplied in a short time while maintaining the diameter of the distal end of the insertion portion 5 without increasing the size.

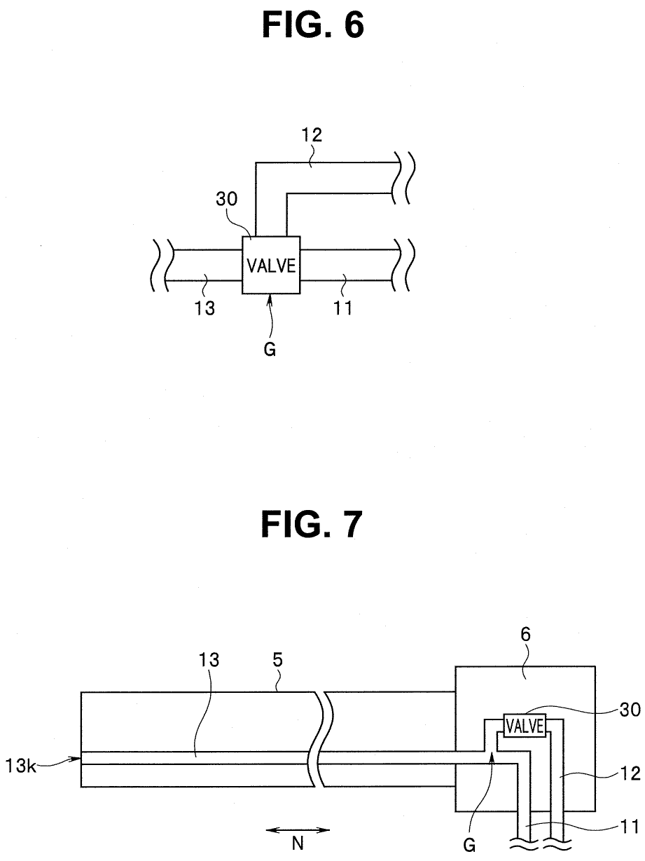

A modification will be described below with reference to FIG. 6. FIG. 6 is a view schematically showing a modification in which the valve of FIG. 1 is provided at the joining portion.

As described above, the case is described in the present embodiment in which the valve 30 is provided in the second flow path 12 to perform a switching operation of flowing and cutoff of the air A.

Regardless of such a case, even when the valve 30 may be provided at the joining portion G as shown in FIG. 6 to come into contact with the liquid R and adjust the timing and ratio of the air A entering the liquid R, the same effects as the effects of the present embodiment described above can be obtained.

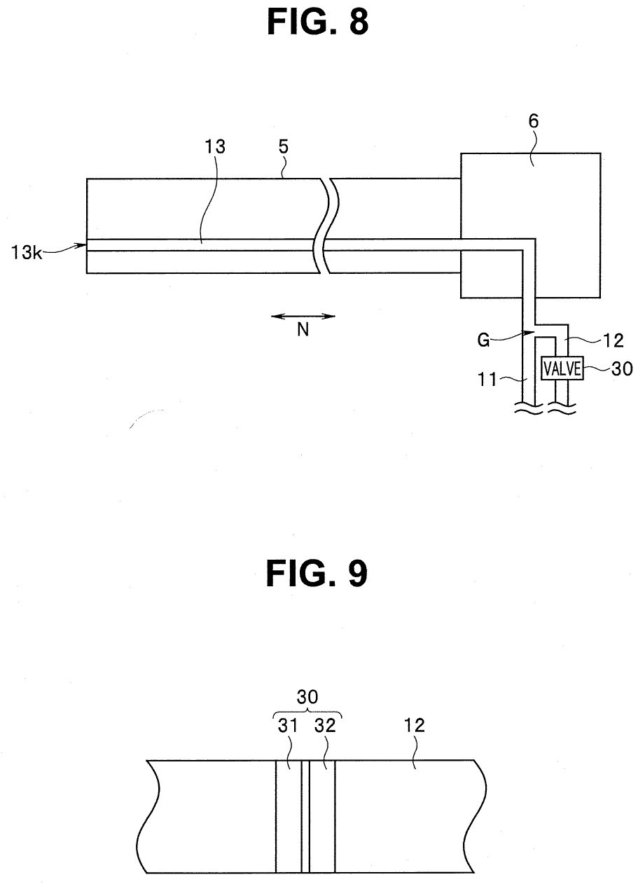

Other modifications will be described below with reference to FIGS. 7 and 8. FIG. 7 is a view schematically showing a modification in which the joining portion of FIG. 1 is provided in the operation portion, and FIG. 8 is a view schematically showing a modification in which the joining portion of FIG. 1 is provided outside the endoscope.

The case is described in the present embodiment described above in which the joining portion G is provided in the flexible tube portion 4.

Regardless of such a case, the joining portion G may be provided in the operation portion 6 as shown in FIG. 7, or may be provided outside the endoscope 1 as shown in FIG. 8.

In this case, as shown in FIGS. 7 and 8, a part of the third flow path 13, and the first flow path 11 and the second flow path 12 are provided outside the insertion portion 5.

According to such a configuration, since the joining portion G is separated from the opening 13k compared with the configuration of the present embodiment described above, the collision force of the mass of the liquid R with respect to the residue S is reduced. However, on the other hand, since only the third flow path 13 is provided in the insertion portion 5, the diameter of the insertion portion 5 can be reduced compared with the configuration of the present embodiment described above.

Even in the configurations shown in FIGS. 7 and 8, the valve 30 may be provided at the joining portion G.

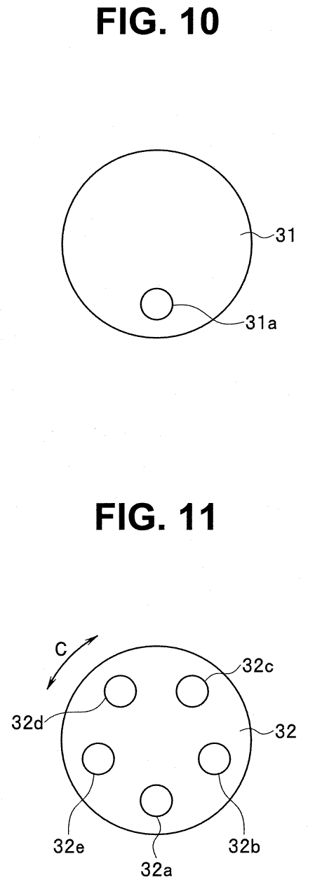

In addition, other modifications will be described below with reference to FIGS. 9 to 11. FIG. 9 is a view schematically showing a modification in which the valve of FIG. 1 is formed by two disks, and the second flow path, FIG. 10 is an enlarged plan view of a fixed disk of FIG. 9, and FIG. 11 is an enlarged plan view of a rotating disk of FIG. 9.

As shown in FIG. 9, the valve 30 may include a fixed disk 31 located on a downstream side and a rotating disk 32 locating adjacent to an upstream side of the fixed disk 31 in the second flow path 12.

As shown in FIG. 10, the fixed disk 31 is formed with one hole 31a penetrating in the longitudinal direction N. As shown in FIG. 11, the rotating disk 32 is formed with a plurality of holes 32a to 32e having a set interval in a rotating direction C and penetrating in the longitudinal direction N, respectively. The number of holes formed in the rotating disk 32 is not limited to five as shown in FIG. 11.

The fixed disk 31 is fixed to the second flow path 12 without moving in the rotating direction C and the longitudinal direction N, and the rotating disk 32 is configured not to move in the longitudinal direction N but to be rotatable in the rotating direction C. The rotating disk 32 rotates under the operation control of the processor 53.

In the valve 30 having such a configuration, when any one of the holes 32a to 32d faces the hole 31a due to rotation, the air A flows to the downstream side of the valve 30, and when any one of the holes 32a to 32d does not face the hole 31a due to rotation, the flowing of the air A to the downstream side of the valve 30 is cut off.

In other words, the holes 32a to 32d repeatedly face and do not face the hole 31a due to the rotation of the rotating disk 32.

As a result, since the air A is intermittently supplied to the joining portion G, the same effect as the effect in the opening/closing valve structure described above can be obtained.

In this case, the ratio of the air A to be mixed with the liquid R is adjusted by a rotation speed of the rotating disk 32 and the number of holes formed in the rotating disk 32.

Other components and effects are similar to the components and effects of the present embodiment described above.

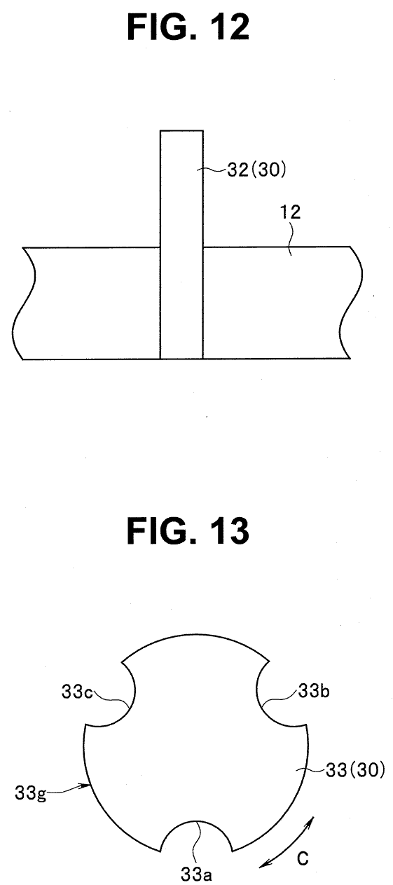

In addition, other modifications will be described below with reference to FIGS. 12 and 13. FIG. 12 is a view schematically showing a modification in which the valve of FIG. 1 is formed only by the rotating disk of the FIG. 11, and the second flow path, and FIG. 13 is a plan view showing a modification of the rotating disk of FIG. 11.

As shown in FIG. 12, the valve 30 may be formed only by the rotating disk 32.

In this case, when the rotating disk 32 is formed to protrude from the second flow path 12, and any one of the holes 32a to 32d is located in the second flow path 12 as the rotating disk rotates, the same effects as the effects of the configurations shown in FIGS. 9 to 11 can be obtained.

Further, as shown in FIG. 13, even when a rotating disk 33 is formed with notches 33a to 33c having a set interval on an outer peripheral surface 33g in the rotating direction C and penetrating in the longitudinal direction N instead of the plurality of holes, the same effect as the effect of the hole shown in FIG. 11 can be obtained.

Needless to say, the number of notches is not limited to three as shown in FIG. 13.

Further, even when the notches are formed, the ratio of the air A to be mixed with the liquid R is adjusted by a rotation speed of the rotating disk 33 and the number of notches formed in the rotating disk 33.

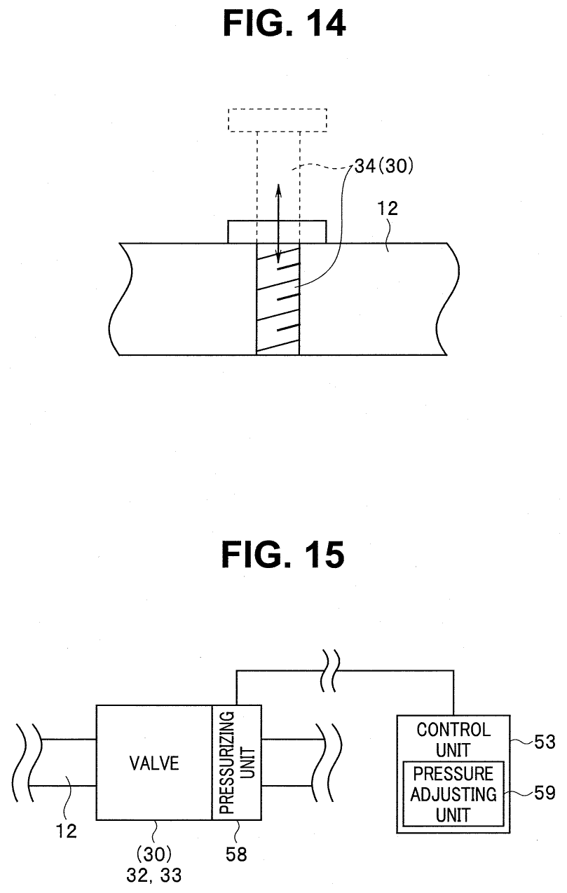

Further, another modification will be described below with reference to FIG. 14. FIG. 14 is a view schematically showing a modification in which the valve of FIG. 1 is configured to open and close by moving up and down with respect to the second flow path 12, and the second flow path.

As shown in FIG. 14, even when the valve 30 may be formed by, for example, a screw type valve 34 that can advance and retract to and from the second flow path 12 by moving up and down with respect to the second flow path 12, the same effect as the effect of the present embodiment described above can be obtained.

Although various modifications of the configuration of the valve 30 are shown in FIGS. 9 to 14, regardless of the modifications, other configurations may be naturally used as long as the second flow path 12 can be intermittently cut off

For example, the valve 30 may be formed by a valve that intermittently crushes and closes the second flow path 12.

In addition, the valve 30 may be provided in the air feeding apparatus 52 and may be configured to intermittently flow the air A through the second flow path 12.

Further, another modification will be described below with reference to FIG. 15. FIG. 15 is a view schematically showing a modification in which the valve of FIG. 1 is provided with a pressurizing unit, and the second flow path and the processor.

As shown in FIG. 15, for example, a pressurizing unit 58 may be provided in the valve 30 to pressurize the air A flowing from the air feeding apparatus 52 to the second flow path 12 under the operation control of the processor 53 and give acceleration.

An example of the pressurizing unit 58 may include a windmill that gives acceleration to the rotation of the rotating disks 32 and 33 described above. The windmill may be provided in the air feeding apparatus 52.

In addition, the pressurizing unit 58 may be configured to increase a supply pressure in the air feeding apparatus 52.

In this case, an example of the pressurizing unit 58 includes a compression apparatus such as a peristaltic pump, a compression air tank, or a supercharger that uses the energy of the air feeding apparatus 52 to increase the pressure of the air.

Further, a pressure adjusting unit 59 may be provided in the processor 53 to adjust the pressure applied from the pressurizing unit to the air A flowing from the air feeding apparatus 52 to the second flow path 12.

In this way, the air A may be intermittently and strongly mixed into the liquid R using the pressurizing unit 58.

### Second Embodiment

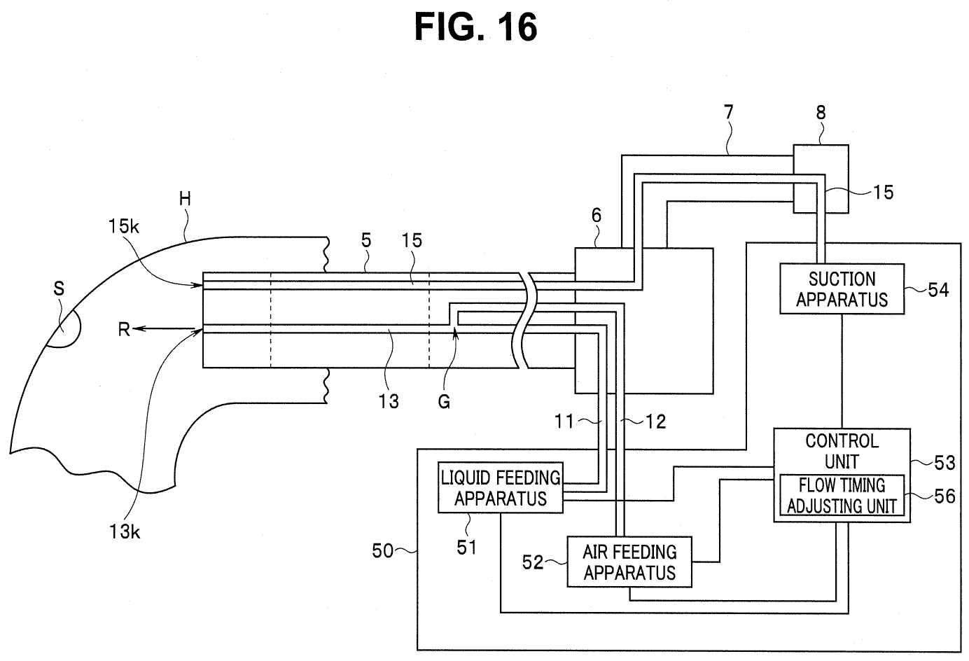

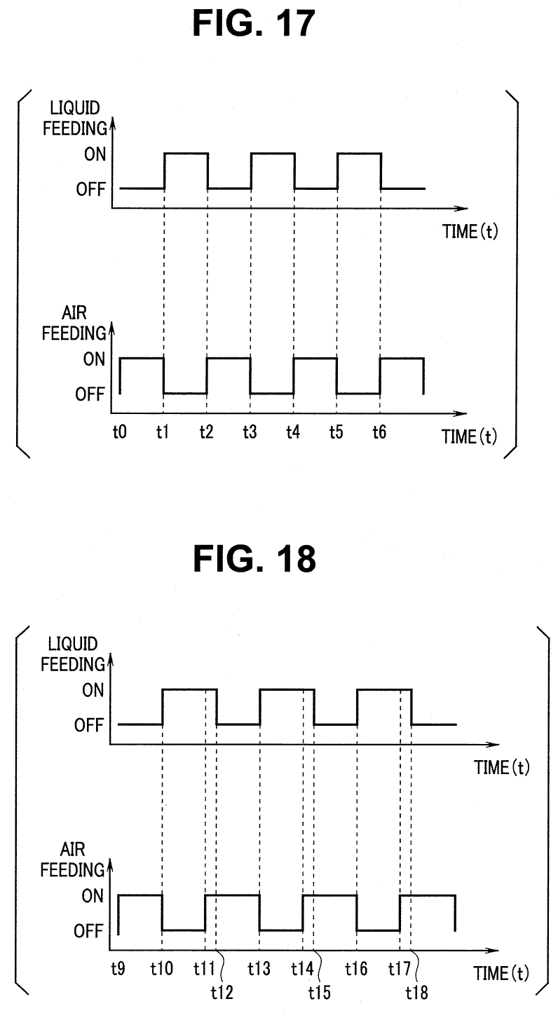

FIG. 16 is a view schematically showing a configuration of an endoscope apparatus of a second embodiment, and FIG. 17 is a chart showing liquid feeding and water feeding timing by a flow timing adjusting unit in FIG. 16.

The configuration of the endoscope apparatus of the second embodiment is similar to the configuration of the endoscope apparatus of the first embodiment shown in FIGS. 1 to 5 described above except that the valve body is not provided and a fluid timing adjusting portion controls timing of the liquid feeding apparatus and the air feeding apparatus to alternately flow the liquid R and the air A in layers to the third flow path 13 from the first flow path 11 and the second flow path 12 in the joining portion G.

Therefore, only the differences will be described, and the components similar to the components in the first embodiment are denoted by the same reference numerals and will not be described.

As shown in FIG. 16, the processor 53 may include a flow timing adjusting unit 56 electrically connected to the liquid feeding apparatus 51 and the air feeding apparatus 52.

Under the operation control of the processor 53, the flow timing adjusting unit 56 adjusts a time during which the liquid R flows from the first flow path 11 to the third flow path 13 through the joining portion G and a time during which the air A flows from the second flow path 12 to the third flow path 13 through the joining portion G, and thus the liquid R and the air A alternately flow in layers in the third flow path 13.

Specifically, as shown in FIG. 17, in such a manner that after air feeding is performed by the air feeding apparatus 52 from a time t0 to a time t1, the air feeding is stopped at the time t1, liquid feeding is performed by the liquid feeding apparatus 51 from the time t1 to a time t2, the liquid feeding is stopped at the time t2, the air feeding is performed again by air feeding apparatus 52 from the time t2 to a time t3, the air feeding is stopped at the time t3, the liquid feeding is performed by the liquid feeding apparatus 51 from the time t3 to a time t4, and the liquid feeding is stopped at the time t4 . . . , for example, in such a manner that the air feeding and the liquid feeding are alternately repeated from the time t0 to a time t6, the flow timing adjusting unit 56 performs switching of supply timing control of the liquid feeding apparatus 51 and the air feeding apparatus 52 by a pulse signal.

As a result, the liquid R and the air A alternately flow in layers in the third flow path 13.

Accordingly, in the present embodiment, the valve 30 is not necessary as shown in FIG. 16 unlike the first embodiment described above. Other components are similar to the components of the first embodiment described above.

Even with such a configuration, the same effects as the effects of the first embodiment described above can be obtained.

A modification will be described below with reference to FIG. 18. FIG. 18 is a chart showing a modification of liquid feeding and water feeding timing by the flow timing adjusting unit in FIG. 17.

As shown in FIG. 18, in order to further increase the supply pressure of the liquid R by the air A, the flow timing adjusting unit 56 may turn on the air feeding apparatus 52 at t11 to t12, t14 to t15, and t17 to t18 in liquid feeding ON times t10 to t12, t13 to t15, and t16 to t18.

In other words, the flow timing adjusting unit 56 may control the timing so as to start the air feeding, overlapping with the end of the liquid feeding in the liquid feeding control performed intermittently.

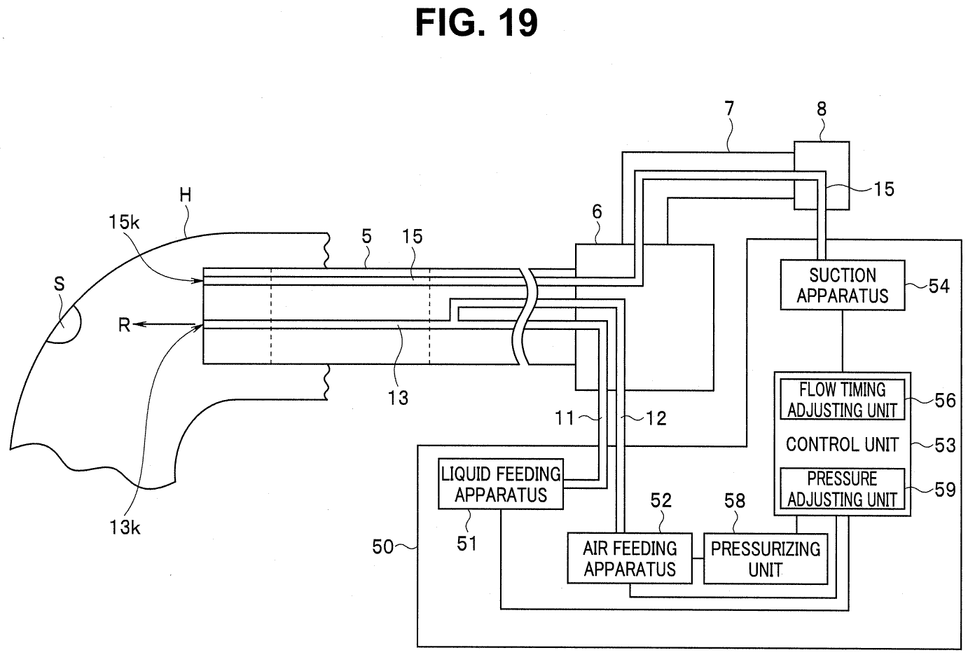

Another modification will be described below with reference to FIG. 19. FIG. 19 is a view schematically showing a modification of the endoscope apparatus in which the pressurizing unit is provided in the external apparatus of FIG. 16.

As shown in FIG. 19, even in the present embodiment, the above-described pressurizing unit 58 subjected to the drive control by the processor 53 may be electrically connected to the air feeding apparatus 52.

In this case, the pressure adjusting unit 59 may also be provided in the processor 53.

In the first and second embodiments described above, under the operation control of the processor 53, the air feeding apparatus 52 can adjust the ratio of the air to be mixed with the liquid R in the joining portion G from 0 to 100% depending on the degree of contamination of the large intestine H, and may be configured to change the timing of the liquid R and the air A ejected alternately from the opening 13k.

Further, as an example, the case is described in the first and second embodiments described above in which the insertion portion 5 is inserted into the large intestine H, but regardless of the case, the present invention may be naturally applied to a case where the insertion portion 5 is inserted into another body cavity to clean the contaminations in the body cavity.

In the first and second embodiments described above, the case is described in which the endoscope apparatus 100 is a medical endoscope apparatus having a function of removing the residue adhering to the large intestine, but regardless of the case, the present invention may also be naturally applied to an industrial endoscope apparatus having a function of removing contaminations strongly adhering to the inside of the conduit.

According to the present invention, it is possible to provide an endoscope apparatus having a configuration capable of removing the contaminations with a small amount of fluid to be supplied in a short time while maintaining the diameter of the distal end of the insertion portion without increasing the size.

Furthermore, the present invention is not limited to the embodiments described above, and various changes may be appropriately made without departing from a gist or spirit of the invention which can be read from the claims, the entire specification, and the drawings.

What is claimed is:

1. An endoscope apparatus comprising:
an insertion portion configured to be inserted into a subject from a distal end side in a longitudinal direction;
a liquid feeding apparatus configured to cause a liquid to flow in a first flow path;
an air feeding apparatus configured to cause air to flow in a second flow path;
a third flow path provided at least partially in the insertion portion and configured to cause the first flow path and the second flow path to communicate with each other;
a joining portion in which the air flowing in the second flow path intermittently flows to the liquid flowing in the first flow path with a timing in which the air is not mixed with the liquid, the joining portion of the first flow path and the second flow path being formed in the third flow path;
a processor configured to control an operation of the liquid feeding apparatus and the air feeding apparatus such that the liquid and the air flow alternately in the third flow path through the joining portion;
a distal end portion provided at a distal end of the insertion portion in the longitudinal direction and including an opening from which the liquid and the air flowing alternately in the third flow path are ejected into the subject in the longitudinal direction; and
a valve configured to perform a switching operation of flowing and cutoff of the air in the second flow path under operation control of the processor such that the liquid and the air alternately flow in the third flow path.

2. The endoscope apparatus according to claim 1, further comprising an operation portion connected to a proximal end of the insertion portion, wherein
the valve is provided in the operation portion.

3. The endoscope apparatus according to claim 1, wherein the valve is provided in the insertion portion.

4. The endoscope apparatus according to claim 1, wherein the valve is provided in the second flow path between the air feeding apparatus and the operation portion.
